(19) United States
(12) Patent Application Publication
(10) Pub. No.: US 2012/0046618 A1
(43) Pub. Date: Feb. 23, 2012

(54) NEEDLE AND SYRINGE

(76) Inventor: Iden Shams, London (GB)

(21) Appl. No.: 13/263,885

(22) PCT Filed: Apr. 9, 2010
(86) PCT No.: PCT/GB2010/000736

§ 371 Date: Oct. 11, 2011

(30) Foreign Application Priority Data
Apr. 9, 2009 (GB) 0906195.3

(51) Int. Cl. A61M 5/32

(52) U.S. Cl. 604/241

(57) ABSTRACT

An intrathecal needle having a tapered male connector, and preferably with an external diameter of less than 4 mm. Also provided are syringes having a female tapered connector having a maximum internal diameter of 4 mm.

### FIELD OF THE INVENTION

The invention relates to needles and syringes for intrathecal administration of medicaments.

### BACKGROUND AND PRIOR ART

Hypodermic needle and syringe assemblies are known, in which a hollow injection needle is fitted with a connector, for connection to a syringe, or other such collection device such as an evacuated container. The construction of the connector on the needle and the syringe must be such as to provide a fluid-tight seal when connected.

There are a few standard connection systems in the medical device field, and these allow for interoperability of needles, syringes and so forth. In common use is the Luer standard, defined in e.g. British Standard BS EN 1707:1977. In essence, the standard defines tolerances for the dimensions and configuration of these essentially tapered connections, having a taper of 6%. Three basic types of Luer connection are used: Luer Slip and Luer Lock connections are “male” connections providing an externally tapered tubular connection, the Luer Lock standard further incorporating a cylindrical hub surrounding the tapered tube, and having an internal thread. Female Luer connections provide a tubular connection having a tapered bore, and may be provided with protruding lugs on the outside of the tubular connection, to engage with a corresponding internal thread of a male Luer Lock connector.

Different sized needles are fitted with identical Luer connections, to allow interoperability between, e.g. needles of different length and bore, and syringes of different capacities.

However, this interoperability can lead to danger. Certain medicaments are formulated, or licensed solely for particular administration routes, and their use in other ways may prove dangerous, if not fatal. One such example is the class of medicaments formulated for intrathecal administration, i.e. for injection into the spinal canal. Such injections may be used, e.g. for spinal anaesthesia, chemotherapy, or pain management. Drugs to be administered intrathecally must be specially formulated at the correct concentration, and are often free of other substances that can be used for e.g. intravenous injection such as preservatives. Thus, medicaments for intrathecal injection and also for intravenous injection may be prepared in identical syringes, leading to the possibility of the wrong formulation being used in intrathecal administration with serious negative consequences. It is among the objects of the present invention to attempt a solution to this problem.

### SUMMARY OF THE INVENTION

Accordingly, the invention provides an intrethecal injection needle comprising a hypodermic needle in fluid connection with a connector comprising a tube having a tapered outer surface decreasing in diameter from the end of the tube proximal to said hypodermic needle towards the end of the tube distal from said hypodermic needle, and a hub, coaxially disposed around said tube, said hub having an internal thread. The provision of this thread allows such an intrathecal needle to be positively locked onto a corresponding syringe. This may preferably be achieved by arranging the internal thread such that it surrounds at least a portion of the tapered tube.

By utilising a “male” tapered connection tube on a needle, this prevents attachment of the needle to the vast majority of syringes intended for other modes of administration, as these syringes are almost always produced with male connectors. The only exceptions to this are enteral syringes, designed for delivering material into e.g. the stomach via an enteral feeding tube.

This system, having an injection needle connected to a male tapered connector, thereby provides a unique needle hub, for the first time provides built-in incompatibility between intrathecal needles utilising the male hub and syringes, especially of the pre-loaded variety, containing medication intended for administration by a different route. By overturning the normal design driver of compatibility, a new and safer needle assembly is provided.

It is preferred that said tube has a maximum outside diameter of less than 4 mm. Standard “Luer” connectors have diameters ranging from 4.2 to 4.7 mm. By choosing the dimensions of the tapered connection to be less than the Luer standard, this prevents interconnection of e.g. pre-filled syringes intended for administration by other routes such as intravenous, intramuscular or subcutaneous injection being administered by the intrathecal route. This also makes the needle incompatible with enteral syringes, having a standard Luer diameter of between 4.2 and 4.7 mm.

It will be appreciated that the tube and hub of the connector may ideally and preferably be made from plastics material of a generally rigid form to as to allow proper operation of the connection and screw thread.

In a second aspect the invention provides a syringe for connection to an intrathecal injection needle having a tube with a maximum outside diameter of less than 4 mm, said syringe comprising a syringe body in fluid connection with a tube having a tapered bore increasing in diameter from the end of the tube proximal to said syringe body towards the end of the tube distal from said syringe body, and having an maximum bore diameter of less than 4 mm.

The invention also provides a syringe according to this third aspect, and further comprising projecting lugs located on the outside of the tube adjacent the end of the tube distal from the syringe body, and adapted to cooperate with the internal thread of a needle according to the second aspect of the invention.

The scope of the invention also includes an intrathecal injection needle substantially as described herein, with reference to and as illustrated by any appropriate combination of the accompanying drawings.

The scope of the invention also includes a syringe substantially as described herein, with reference to and as illustrated by any appropriate combination of the accompanying drawings.

### BRIEF DESCRIPTION OF THE DRAWINGS

The invention will be described with reference to the accompanying drawings, in which:

FIG. 1 is a perspective view of an intrathecal needle of the present invention;

FIG. 2 is a cut-away perspective view of an intrathecal needle of the present invention;

FIG. 3 is a perspective view of a syringe according to the present invention;

FIG. 4 is a cross-sectional view of part of a syringe according to the present invention; and

FIG. 5 is a cross-sectional part view of an intrathecal needle of the present invention attached to a syringe according to the present invention.

### DESCRIPTION OF PREFERRED EMBODIMENTS

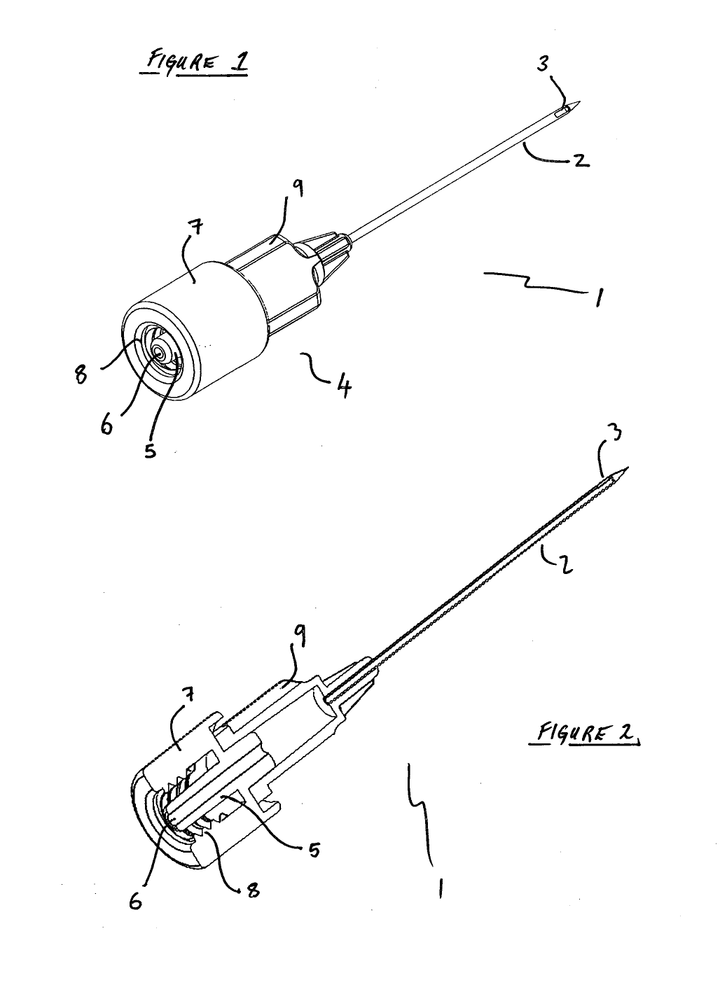

FIG. 1 illustrates, in perspective view, an intrathecal injection needle, generally indicated by 1. The unit comprises a hypodermic needle 2 having a hollow bore with an outlet port 3 adjacent the sharpened end of the hypodermic needle 2. The hypodermic needle is in fluid connection with a connector 4 comprising a tube 5 having a tapered outer surface. The tube has a bore of 1.2 mm. The outside diameter of the tube decreases from the end of the tube proximal to the hypodermic needle 2 towards the open end 6 of the tube. In this embodiment, the outside diameter of the tube decreases linearly from 3.19 mm to 2.82 mm over a length of 8 mm. Surrounding the tube 5 in the embodiment is a hub 7, having the form of a cylindrical tube, coaxially disposed around the tube 5. In the internal surface of the hub 7 is an internal thread 8. By preference, the thread 8 is of a double start form, to enable rapid connection to a corresponding syringe, described below, and to prevent cross-threading. A gripping portion 9 in the form of externally-projecting fins is also provided. The connector, comprising the hub, tube and gripping portions is preferably made as a single piece plastics moulding. In preferred embodiments, the maximum outside diameter of the tube is less than 4 mm, more preferably less than 3.5 mm, and most preferably less than 3 mm. In this way, interconnection with “standard” Luer connections (which have outside diameters ranging from 4.2-4.7 mm) is prevented.

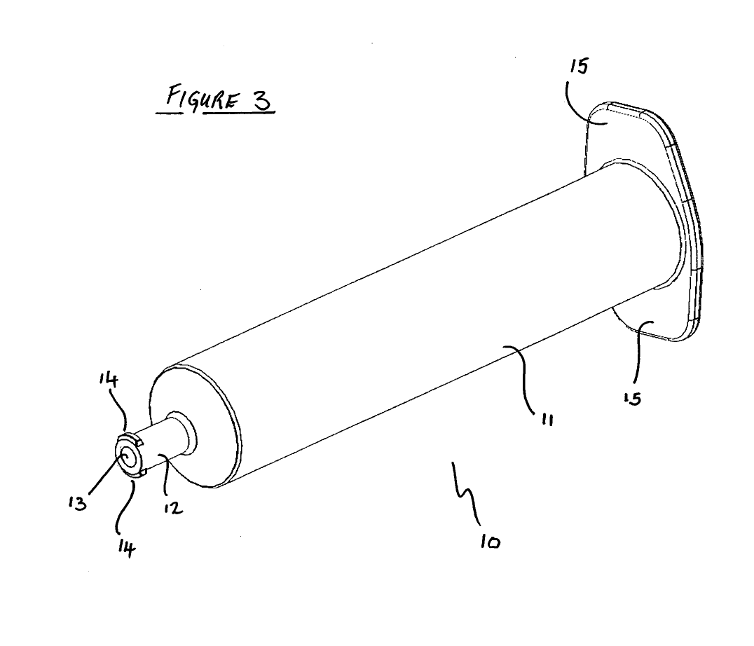

FIG. 2 shows, in cut-away perspective view, an intrathecal injection needle, generally indicated by 1. This view more clearly illustrates the arrangement of hollow hypodermic needle 2, in fluid connection with the externally tapered tube 5. The internal threads 8 of the hub 7 are visible in this cut-away view.

FIG. 3 shows, in perspective view, a syringe according to the present invention, generally indicated by 10, and suitable for connection to an intrathecal needle of the present invention. The syringe comprises a hollow syringe body 11 in fluid connection with a tubular portion 12, having a hollow bore 13. The bore 13, defined by the walls of the tubular portion 12, is of a tapered construction with an internal diameter increasing from the end of the tube 12 proximal to the syringe body 11 towards the open end of the tube distal to the syringe body. The internal taper of the tubular portion 12 is chosen to match that of the external taper of a needle connector with which the syringe in to be used.

Projecting from the outside of the syringe tube 12 are two lugs 14 that are so-sized as to cooperate with the internal thread 8 of a needle connector, as illustrated in FIGS. 1 and 2. Located at the other end of the syringe body 11 is a pair of projecting wings 15 to facilitate the expulsion of fluid contained with the syringe body by use of a cooperating syringe plunger (not illustrated) in a conventional manner.

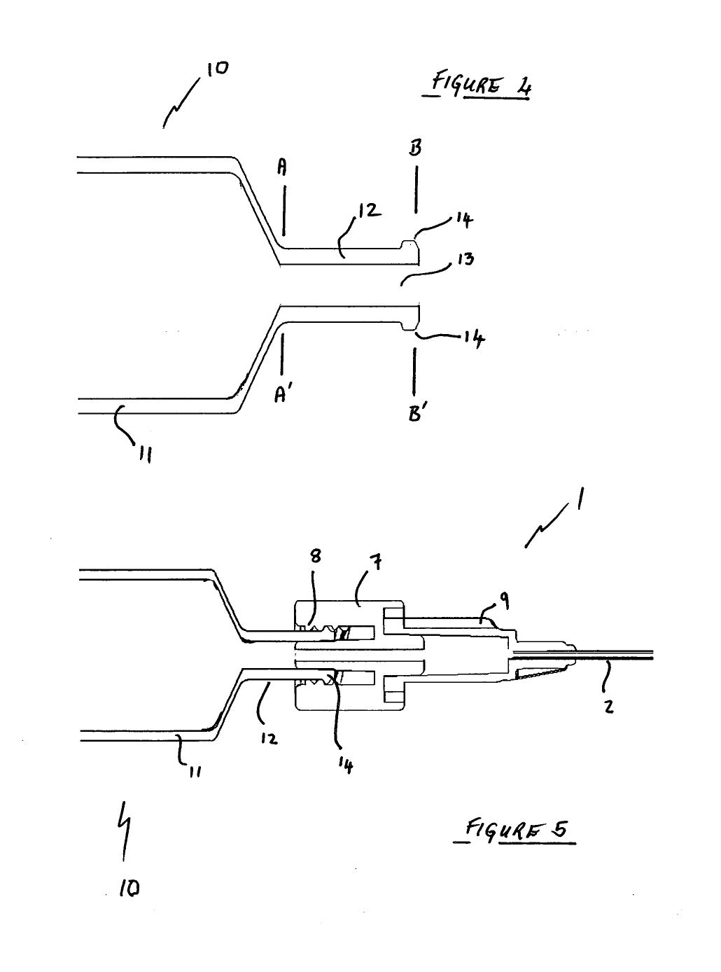

FIG. 4 shows cross-sectional view of part of a syringe 10 according to the present invention. The figure illustrates a portion of the syringe body 11 fluidly connected to the tubular portion 12. The internal bore 13 of the tube is tapered such that the internal diameter of the bore increases from the end of the tube 12 proximal to the syringe body 11, indicated by the section line A-A′ to the open end of the tube 12 distal from the syringe body 11, indicated by the section line B-B′. In this embodiment, the internal diameter of the bore a the position A-A′ is 2.82 mm, and at B-B′ is 3.19 mm. A taper of approximately 6% is particularly preferred.

The projecting lugs 14 are also illustrated in FIG. 4.

FIG. 5 illustrates, in cross-sectional view, a portion of a syringe 10 in accordance with the invention, and a portion of an intrathecal injection needle 1, connected to each other. The features of each component are as numbered above, and illustrated in corresponding FIGS. 1-4.

1-6. (canceled)

7. An apparatus for use in a method of intrathecal administration of a medicament, said apparatus comprising:
an intrathecal injection needle comprising a hypodermic needle in fluid connection with a connector comprising a tube having a tapered outer surface decreasing in diameter from the end of the tube proximal to said hypodermic needle towards the end of the tube distal from said hypodermic needle, and a hub, coaxially disposed around said tube, said hub having an internal thread; and
a syringe, connectable to said intrathecal injection needle, said syringe comprising a syringe body in fluid connection with a tube having a tapered bore increasing in diameter from the end of the tube proximal to said syringe body towards the end of the tube distal from said syringe body;
wherein said tube of said intrathecal injection needle has a maximum outside diameter of less than 4 mm; and
wherein said syringe has a maximum bore diameter of less than 4 mm.

8. An apparatus according to claim 7, wherein said syringe further comprises projecting lugs located on the outside of the tube adjacent the end of the tube distal from the syringe body, and configured to cooperate with the internal thread of said intrathecal injection needle.

9. An apparatus according to claim 8, wherein the internal thread of said needle connector surrounds at least a portion of the connector's tapered tube.

10. An apparatus according to claim 9, wherein the hub of said needle further comprises a gripping portion, and said hub, gripping portion and tube is made as a single piece plastics moulding.

11. An apparatus according to claim 8, wherein the hub of said needle further comprises a gripping portion, and said hub, gripping portion and tube is made as a single piece plastics moulding.

12. An apparatus according to claim 7, wherein the internal thread of said needle connector surrounds at least a portion of the connector's tapered tube.

13. An apparatus according to claim 12, wherein the hub of said needle further comprises a gripping portion, and said hub, gripping portion and tube is made as a single piece plastics moulding.

14. An apparatus according to claim 7, wherein the hub of said needle further comprises a gripping portion, and said hub, gripping portion and tube is made as a single piece plastics moulding.
